(19) United States
(12) Patent Application Publication
(10) Pub. No.: US 2003/0118916 A1
(43) Pub. Date: Jun. 26, 2003

(54) Heteroaromatic compounds having two-photon absorption activity

(76) Inventors: Giorgio Pagani, Milano (IT); Alessandro Abbotto, Milano (IT); Renato Bozio, Selvazanno Destro (IT)

Correspondence Address: JACOBSON HOLMAN PLLC, 400 SEVENTH STREET N.W., SUITE 600, WASHINGTON, DC, 20004, US

(21) Appl. No.: 10/221,195

(22) PCT Filed: Dec. 22, 2000
(86) PCT No.: PCT/EP00/13193

(51) Int. Cl. G03C 1/00; C 07D 4 1/14; C07D 417/14

(52) U.S. Cl. 430/2; 548/181; 546/102; 546/152; 546/256

(57) ABSTRACT

The present invention relates to new heteroaromatic compounds having two-photon absorption activity. According to the invention, said compounds are suitable for use as optical power limiting agents via two-photon absorption or for use as imaging agents with two-photon absorbing activity for application in two-photon laser scanning confocal fluorescence microscopy. Compositions including said compounds and intermediates for their preparations are also within the scope of the present invention.

It is known that molecular systems interact with the electromagnetic radiation through parametric or dissipative processes. In parametric processes, energy and moment undergo exchange among the different modes of the field. In dissipative processes, energy absorption and emission between the molecules and the field is observed. The two-photon absorption is a dissipative process.

In the presence of an intense light radiation (laser), organic molecules can show a two-photon absorption. This nonlinear optical process can be described as the simultaneous absorption of two photons having the same frequency ω. As a results, the molecule goes from its ground state S₀ to its excited state S₂, via a virtual intermediate state i. The system can then decay to its lower energy singlet excited state S₁ through non-radiative mechanisms. The rate of two-photon absorption scales quadratically with the intensity I of the incident laser radiation, whereas the single-photon absorption scales linearly.

Optical power limiting is becoming a field of increasing interest in applications such as protection of human eyes and optical sensors against intense laser radiation exposure. An ideal optical limiter is a system which is completely transparent up to a certain threshold of intensity level of the incident radiation. As a consequence, the transmitted intensity Iₜ is the same as the incident intensity I₀. In contrast, at high intensities the transmitted intensity levels off and becomes independent on the radiation intensity. Large two-photon absorption cross-sections σ₂ are required in order to have efficient optical limiters working via a two-photon absorption mechanism. However, many molecules known so far have weak two-photon absorption activity, which limit their applicability in optical limiting devices. The guidelines followed in the present invention for providing new chromophores with enhanced activity are based on highly conjugated and polarizable π systems, usually associated with large σ2 values. Molecules having large two-photon absorption cross-sections are in great demand for a variety of applications, including two-photon confocal laser scanning fluorescence microscopy (Denk, W.; Strickler, J. H.; Webb, W. W. Science, 1990, 248, 73-76), optical limiting (Ehrlich, J. E.; Wu, X. L.; Lee, I.-Y. S.; Hu, Z.-Y.; Roeckel, H.; Marder, S. R.; Perry, J. W. Opt. Lett. 1997, 22, 1843-1845), three-dimensional optical data storage (Strickler, J. H.; Webb, W. W. Opt. Lett. 1991, 16, 1780), three-dimensional imaging of biological systems (Gura, T. Science 1997, 256, 1988-1990) and organic coatings (Reinhardt, B. A.; Brott, L. L.; Clarson, S. J.; Dillard, A. G.; Bhatt, J. C.; Kannan, R.; Yuan, L. X.; He, G. S.; Prasad, P. N. Chem. Mat. 1998, 10, 1863-1874), and photodynamic therapy (Stiel, H.; Teuchner, K.; Paul, A.; Freyer, W.; Leupold, D. J. Photochem. Photobiol. A: Chem. 1994, 80, 289).

For optical power limiting applications, nonlinear optical materials showing two-photon absorption have the great advantage, with respect to other optical limiters, to possess a high transmissivity at low-intensity fundamental optical frequencies, which are much smaller than the linear absorption frequency.

In accordance with the present invention, new active molecules are provided for two-photon absorption materials with excitation by a near-infrared laser radiation, that is in a spectral region where most organic and, particularly, biological materials show a very high optical transparency.

In accordance with the present invention, compounds are provided having the following general formula (I)

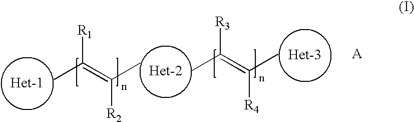

wherein Het-1 and Het-3 may be the same or different, and are selected among the following heterocyclic groups:

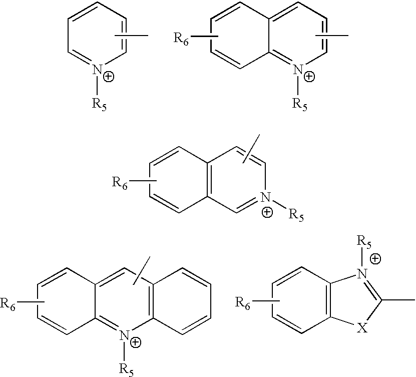

wherein X may be O, S or Se, and wherein R₅ and R₆ are the same or different, and are selected from the group consisting of H, alkyl groups having from 1 to 18 carbon atoms, alkoxy, aminoalkyl, alkylhalide, hydroxyalkyl, alkoxyalkyl, alkylsulfide, alkylthiol, alkylazide, alkylcarboxyclic, alkylsulfonic, alkylisocyanate, alkylisothiocyanate, alkylalkene, alkylalkyne, aryl, and which can contain electronpoor ethenylic moieties such as maleimide, capable to react with nucleophilic groups such as —SH;

and Het-2 is selected among the following heterocyclic groups:

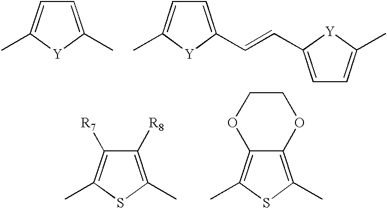

wherein Y may be O, S, NZ, wherein Z=H, lower alkyl, aryl; and R₇ and R₈ may be the same or different, and are lower alkyl, lower alkoxy or hydroxyalkyl;

wherein n=1, 2, and A is selected among the anions alkylsulfonate, arylsulfonate, triflate, halide, sulfate, phosphate;

and wherein R₁, R₂, R₃, R₄, the same or different, are indipendently selected from the group of H, lower alkyl, alkoxyalkyl, aryl, cyano, alkoxycarbonyl, —(CR₉R₁₀)ₘ-Het, wherein 0<m<10, R₉ and R₁₀, the same or different, are selected from the group of H, lower alkyl, and Het may be Het-1 or Het-2 or Het-3.

The alkyl group substituted with electronpoor ethenylic groups, as above defined, is preferably referred to, but not limited to, maleimide.

For the uses according to the present invention, the above compounds can be utilised as such or prepared in suitable compositions, such as solutions or in the solid state.

In a further aspect of the present invention, compounds having the above general formula (i) are processed into compositions based on polymers or silica-based lattices. Therefore in accordance with the present invention, compositions are also provided including a compound of said general formula (I) and a polymer material which comprises poly(methacrylate), polyimide, polyamic acid, polystyrene, polycarbonate, polyurethane or an organically-modified silica (SiO₂) network;

chromophore-functionalized polymer materials or organically-modified silica (SiO₂) network, prepared by condensation of a chromophore compound of general formula (I) and a polymer material which comprises poly(methacrylate), polyimide, polyamic acid, polystyrene, polycarbonate, polyurethane or an organically-modified silica (SiO₂) network.

In another aspect of the present invention, such compositions or chromophore functionalized materials can be processed as thin films either by a film casting procedure or by spin-dipping or, alternatively, by spin-coating, onto any type of substrate, including silica glass, quartz, silicon.

Features and advantages of the present invention will become readily apparent by reference to the following detailed description, in conjunction with the accompanying drawings, in which:

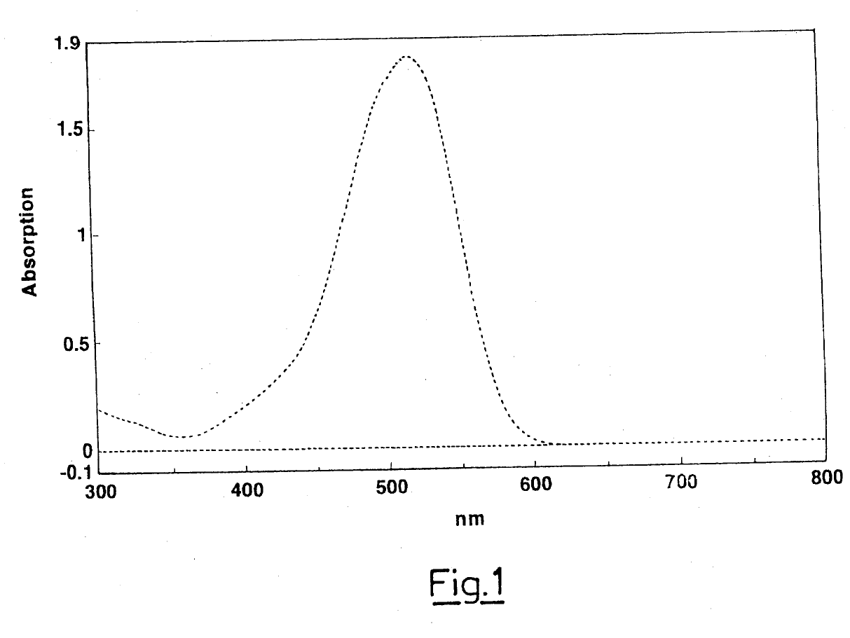

FIG. 1 shows a typical absorption spectrum of a host-guest film of compound (3) in an organically-modified silica (SiO₂) matrix.

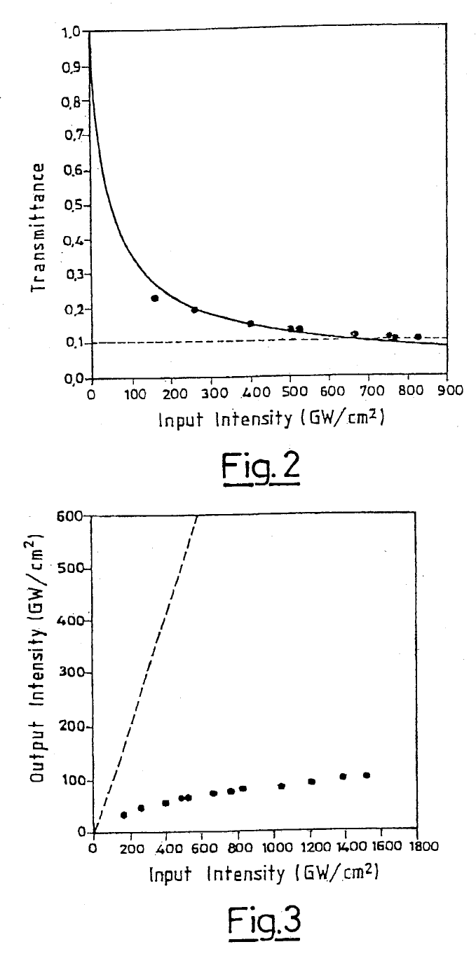

FIGS. 2 and 3 show the transmittance and output intensity, respectively, as a function of the input intensity, typically measured for compound (3) in DMSO (dimethylsulfoxide) solution.

A detailed description of the invention is provided, with reference to certain compounds, which possess a structure corresponding to the formulas defined as (3), (5), (9), (11), and (14), and a chromophore-functionalized material (16), with examples which are not limiting the present invention.

### EXAMPLES

### Example 1

Compound (3), endowed with two photon absorption properties, was prepared starting from compound (1) (Abbotto, A.; Bradamante, S.; Facchetti, A.; Pagani, G. A. J. Org. Chem., 1997, 62, 5755-5765) through a Vilsmeier type reaction, followed by coupling with N-methylpicolinium triflate with catalytic amount of piperidine, according to the following scheme:

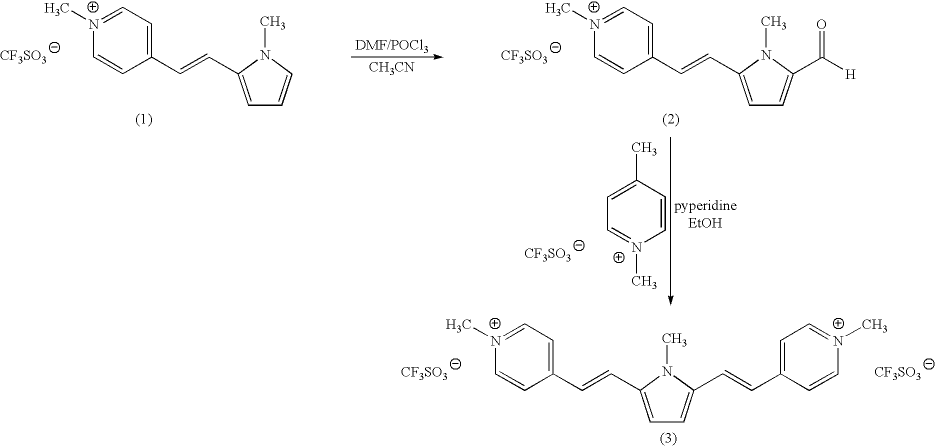

1-(N-methylpyrid-4-yl)-2-(N-methyl -5-formylpyrrol-2-yl)ethylene triflate (2)

Freshly distilled POCl₃ (0.449 g, 2.93 mmol) was added dropwise, at 5° C. under a nitrogen atmosphere, to anhydrous dimethylformamide (0.214 g, 2.93 mmol). A solution of 1-(N-methylpyrid-4-yl)-2-(N-methylpyrrol-2-yl)ethylene triflate (1) (0.700 9, 2.01 mmol) in anhydrous acetonitrile (15 ml) was added dropwise at 5° C., than the reaction mixture was stirred at ambient temperature for 4 h, observing the formation of a precipitate that was filtered off under reduced pressure and washed with an aqueous solution of K₂CO₃. The product was obtained as a yellow solid (0.530 g, 1.41 mmol, 70%): mp (H₂O)=189-191° C. ¹H-NMR (DMSO-d₆) 9.63 (1 H, s), 8.85 (2H, d, J=6.7), 8.27 (2 H, d, J=6.8), 8.01 (1 H, d, J=16.1), 7.50 (1 H, d, J=16.1), 7.14 (1 H, d, J=4.4), 6.99 (1 H, d, J=4.4), 4.24 (3 H, s), 4.08 (3H, s); ¹³C-NMR (DMSO-d6) 180.0 (1 C), 151.2 (1C), 145.0 (2 C), 140.0 (1 C), 135.0 (1 C), 127.0 (1 C), 125.0 (1 C), 123.5 (3 C), 111.0 (1 C), 48.5 (1 C), 31.5 (1 C); ¹⁵N-NMR (DMSO-d₆-relative to liquid ammonia) 184.5, 151.5. Elemental analysis, calcd for C₁₅H₁₅F₃N₂O₄S: C, 47.87%; H, 4.02%; N, 7.44%. Found: C, 47.68%; H, 4.21%; N, 7.80%.

N-methyl-2,5-[1-(N-methylpyrid-4-yl)ethen-2-yl]pyrrole triflate (3)

A solution of N-methyl-4-picolinium triflate ( 0.424 g, 1.65 mmol) and a few drops of piperidine, in ethanol (8 ml), was added to a solution of (2) (0.621 g, 1.65 mmol) in ethanol (12 ml). The reaction mixture was kept under reflux temperature for 8 h, and then cooled to allow separation of the product. The precipitate was filtered off under reduced pressure and washed with 4 ml of absolute ethanol. The product was obtained as a dark solid (0.680 g, 1.13 mmol, 68%): mp 300-304° C. ¹H-NMR (DMSO-d₆) 8.66 (4 H, d, J=6.8), 8.08 (4 H, d, J=6.9), 7.91 (2 H, d, J=15.9), 7.20 (2 H, d, J=15.9), 7.02 (2 H, s), 4.15 (6 H, s), 3.80 (3 H, s); ¹³C-NMR (DMSO-d₆) 152.5 (2 C), 144.5 (4 C), 135.6 (2 C), 128.0 (2 C), 121.2 (2 C), 122.7 (4 C), 113.5 (2 C), 46.5 (2 C), 30.8 (1 C). ¹⁵N-NMR (DMSO-d₆—relative to liquid ammonia) 190.3 (2 N), 153.7 (1 N). Elemental analysis, calcd for C₂₃H₂₃N₃F₆S₂O₆: C, 44.88%; H, 3.77%; N, 6.83%. Found: C, 44.24; H, 3.77%; N, 6.41%.

### Example 2

Compound (5) was obtained by a condensation of compound (2) with N-methyl-2-quinaldinium triflate (4) with catalytic amount of piperidine, according to the following scheme:

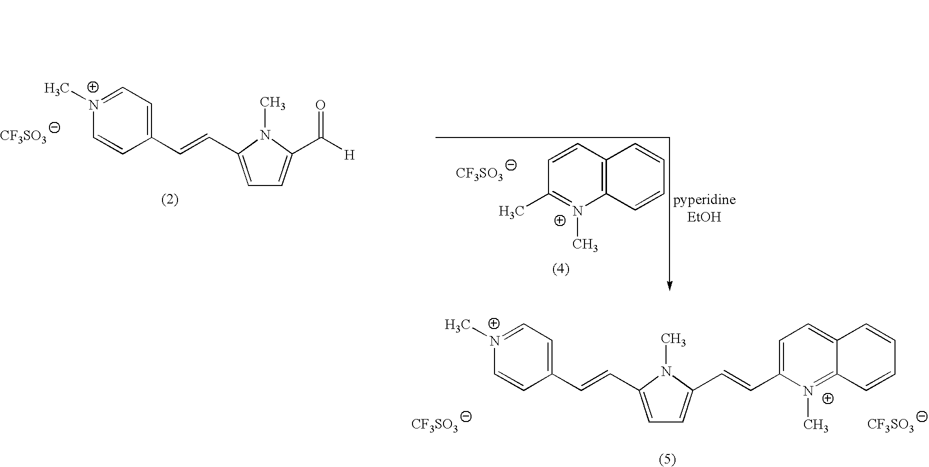

N-methyl-2-quinaldinium trifluoromethansulfonate (4)

A solution of methyltriflate (1.127 g, 7 mmol) in dry benzene (7 ml) was added dropwise to a solution of quinaldine (1.000 g, 6.98 mmol) in 8 ml of the same solvent. The reaction mixture was allowed to react for 2 h at room temperature, then the white precipitate was filtered off under reduced pressure and washed with 2 ml of benzene. The product was obtained as a white solid (2.021 g, 6.64 mmol, 95%): mp 134-135° C. ¹H-NMR (DMSO-d₆) 8.98 (1 H, d, J=8.6), 8.49 (1 H, d, J=9.1), 8.29 (1 H, dd, J=16.9, J=1.2), 8.13 (1 H, m), 8.02 (1 H, d, J=8.6), 7.89 (1 H, t, J=8.5), 4.40 (3 H, s), 3.00 (3 H, s).

N-methyl-2-[1-(N-methylquinol-2-yl)ethen-2-yl]-5-[N-methylpyrid-4-yl)ethen-2-yl]pyrrole triflate (5)

A solution of (4) (0.040 g, 0.137 mmol) and 0.1 ml of piperidine in ethanol (3 ml), was added to a solution of (2) ( 0.050 g, 0.133 mmol) in 4 ml of the same solvent. The mixture was kept under reflux for 2 h and then cooled to allow precipitation of the product. The precipitate was filtered off under reduced pressure and washed with 2 ml of ethanol. The product was obtained as a dark-violet solid ( 0.063 g, 0.095 mmol, 71%): mp 328-329° C. ¹H-NMR (DMSO-d₆) 8.82 (1 H, d, J=9.2), 8.71 (2 H, d, J=6.8), 8.61 (1 H, d, J=9.3), 8.37 (1 H, d, J=9), 8.18 (1 H, d, J=8.2), 8.16 (1 H, d, J=15.4), 8.12 (2 H, d, J=6.8), 8.04 (1 H, m), 7.96 (1 H, d, J=15.7), 7.80 (1 H, d, J=7.6), 7.59 (1 H, d, J=15.2), 7.51 (1 H, d, J=4.56), 7.32 (1 H, d, J =15.9), 7.11 (1 H, d, J=4.5), 4.38 (3 H, s), 4.12 (3 H, s), 3.95 (3 H, s). Elemental analysis, calcd for C₂₇H₂₅N₃F₆S₂O₆: C, 48.72%; H, 3.79%; N, 6.31%. Found: C, 48.55%; H, 3.98%; N, 6-07%.

### Example 3

Compound (7) was prepared by a condensation of 1-methyl-2-pyrrolecarboxaldehyde with N-methyllepidinium triflate (6) with catalytic amount of piperidine. Compound (9) was obtained starting from compound (7) by a Vilsmeier type reaction followed by a condensation with (6), with catalytic amount of piperidine, according on the following scheme:

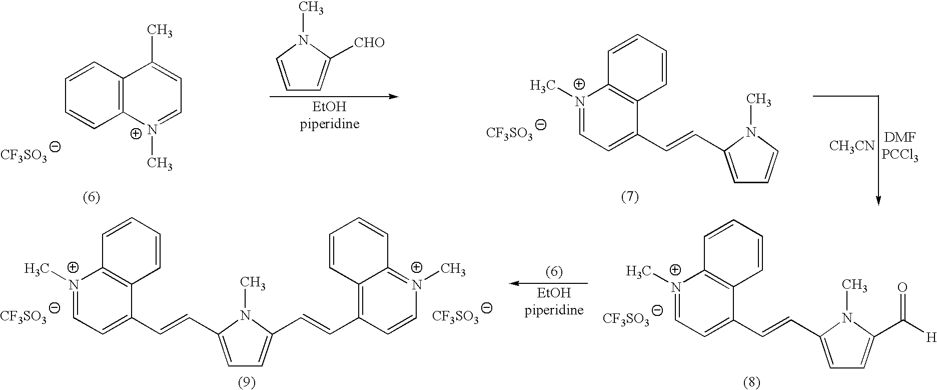

N-methyl-lepidinium trifluoromethansulfonate (6)

A solution of methyltriflate (1.127 g, 7 mmol) in dry benzene (7 ml) was added dropwise to a solution of lepidine (1.000 g, 6.98 mmol) in 8 ml of the same solvent. The reaction mixture was allowed to react for 2 h at room temperature, then the white precipitate was filtered off under reduced pressure and washed with 2 ml of benzene. The product was obtained as a white solid ( 2.100 g, 6.9 mmol, 98.6%). mp 138-140° C.

1-[N-methylquinol-4-yl]-2-(N-methyl-2-pyrrolyl)ethylene triflate (7)

A solution of (6) (1.000 g, 3.3 mmol) and 0.1 ml of piperidine in ethanol (10 ml) was added at room temperature to a solution of N-methyl-2-pyrrolecarboxaldehyde (0.371 g, 3.4 mmol) in 10 ml of the same solvent. The reaction mixture was stirred under reflux for 2h and then cooled in an ice bath. A bright violet precipitate was filtered under reduced pressure and washed with 3 ml of ethanol. The product was obtained as a violet solid (0.850 g, 2.15 mmol, 65%): mp 215-217° C. ¹H-NMR (DMSO-d₆) 9.10 (1 H, d, J=6.7), 8.95 (1 H, d, J=8.5), 8.46 (1 H, d, J=8.8), 8.34 (1 H, d, J=6.7), 8.20 (1 H, t, J=7.45), 8.14 (1 H, d, J=15.45), 7.97 (1 H, t, J=7.7), 7.90 (1 H, d, J=15.45), 7.35 (1 H, d, J=3.95), 7.20 (1 H, m), 6.30 (1 H, m), 4.42 (3 H, s), 3.88 (3 H, m).

1-[N-methylquinol-4-yl]-2-(N-methyl-5-formylpyrrol-2-yl)ethylene triflate (8)

Freshly distilled POCl₃ (0.711 g, 4.64 mmol) was added dropwise, at −15° C. under a nitrogen atmosphere, to anhydrous dimethylformamide (0.339 g, 4.64 mmol), the reaction mixture was diluted with anhydrous acetonitrile (4ml). A solution of 1-(N-methylquinol-4-yl)-2-(N-methylpyrrol-2-yl)ethylene triflate (1) (0.918 g, 2.32 mmol) in anhydrous acetonitrile (15 ml) was added dropwise at −15° C., then the reaction mixture was stirred at ambient temperature for 6 h, observing the formation of a precipitate that was filtered off under reduced pressure. The product was obtained as a red, fluorescent, precipitate (0.530 g, 1.25 mmol, 54%): mp 234-235° C. dec. ¹H-NMR (DMSO-d₆) 9.67 (1 H, s), 9.37 (1 H, d, J=6.3), 9.03 (1 H, d, J=8.45), 8.68 (1 H, d, J=6.45), 8.46 (1 H, d, J=8.65), 8.41 (1 H, d, J=15.45), 8.28 (1 H, t, J=7.8), 8.19 (1 H, d, J=15.45), 8.06 (1 H, t, J=7.55), 7.45 (1 H, d, J=4.35), 7.20 (1 H, d, J=4.30), 4.57 (3 H, s), 4.13 (3 H, s).

N-methyl-2,5-[1-(N-methylquinol-4-yl)ethen-2-yl]pyrrole triflate (9)

A solution of N-methyllepidinium triflate ( 0.146 g, 0.48 mmol) and a few drops of piperidine, in ethanol (8 ml), was added to a solution of (8) (0.200 g, 0.47 mmol) in-ethanol (20 ml). The reaction mixture was kept under reflux for 30 min., and then cooled to allow for separation of the precipitate. The precipitate was filtered off under reduced pressure and washed with 4 ml of absolute ethanol. The product was obtained as a dark blue solid (0.235 g, 0.33 mmol, 70%): mp 311-312° C. ¹H-NMR (DMSO-d₆) 9.22 (2 H, d, J=6.89), 9.02 (2 H, d, J=8.73), 8.60 (2 H, d, J=6.62), 8.39 (2 H, d, J=8.92), 8.27 (2 H, d, J=15.07), 8.25 (2 H, t, J =8.10), 8.19 (2 H, d, J=15.17), 8.02 (2 H, t, J=8), 7.69 (2 H, s), 4.5 (6 H, s), 4.1 (3 H, s); ¹³C-NMR (DMSO-d₆) 151.92 (2 C), 147.08 (2 C), 138.84 (2 C), 137.21 (2 C), 134.78 (2 C), 129.96 (2 C), 128.91 (2 C), 126.13 (2 C), 125.92 (2 C), 119.25 (2 C), 117.86 (2 C), 115.61 (2 C), 114.97 (2 C), 44.28 (2 C), 30.92 (1 C). Elemental analysis, calcd for C₃₁H₂₇F₆N₃O₆S₂: C, 52.03%; H, 3.80%; N, 5.87%. Found: C, 52.20%; H, 4.33%; N, 6.01%.

### Example 4

Compound (11) was prepared by a condensation of compound (8) with bis-2-benzothiazolylmethane (10) (Rai, C.; Braunwarth, J. B. J. Org. Chem. 1961, 26, 3434-3445) in ethanol with catalytic piperidine, according on the following scheme:

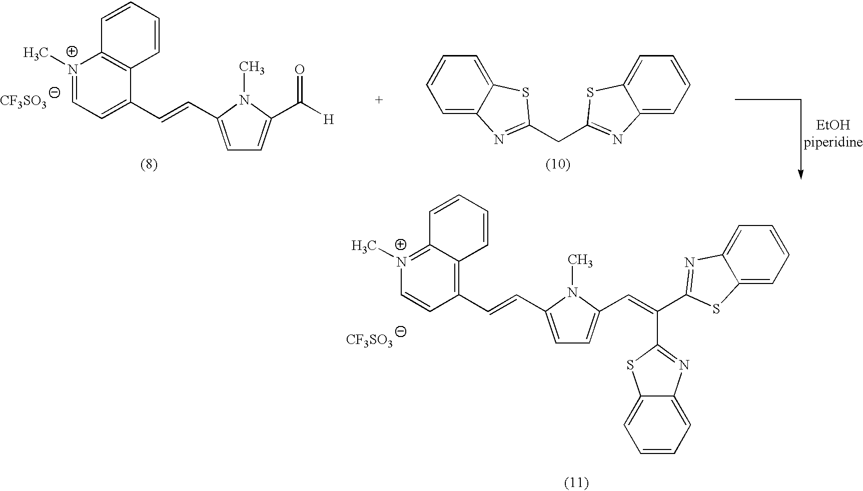

N-methyl-2-[1-(N-methylquinol-4-yl)ethen-2-yl]-5-[1-(bis-2-benzothiazolylmethyl)ethen-2-yl]pyrrole triflate (11)

A solution of (8) (0.118 g, 0.27 mmol) in ethanol (10 ml) was added to a solution of (10) (0.077 g, 0.27 mmol) and 0.1 ml of piperidine in ethanol (8 ml). The mixture was heated under reflux for 30 min and then cooled observing the formation of a bright violet precipitate, that was filtered under reduced pressure and washed with 3 ml of ethanol. The product was isolated as a violet solid (0.090 g, 0.13 mmol, 48.5%): mp 220-221° C. ¹H-NMR (DMSO-d₆) 9.22 (1 H, d, J=6.71), 8.90 (1 H, d, J=8.54), 8.61 (1 H, d, J=6.62), 8.37 (1 H, d, J=8.78), 8.26 (1 H, d, J=7,54), 8.25 (1 H, d, J=15.53), 8.21 (1 H, d, J=7.72), 8.20 (1 H, t, J=7.60), 8.15 (1 H, s), 8.08 (1 H, d, J=7.90), 8.04 (1 H, d, J=15.35), 7.99 (1 H, d, J=8.09), 7.98 (1 H, t, J=7.63), 7.67 (1 H, t, J=7.22), 7.61 (1 H, t, J=7.27), 7.54 (1 H, t, J=7.70), 7.64 (1 H, t, J=7.68), 7.28 (1 H, d, J=4.51), 5.71 (1 H, d, J=4.41), 4.48 (3 H, s), 4.08 (3 H, s). ¹³C-NMR (DMSO-d₆) 166.88 (1 C), 163.93 (1 C), 153.25 (1 C), 152.90 (1 C), 151.96 (1 C), 147.00 (1 C), 138.80 (1 C), 136.02 (1 C), 135.71 (1 C), 134.73 (1 C), 134.67 (1 C), 133.20 (1 C), 128.90 (1 C), 126.87 (1 C), 126.72 (1 C), 126.30 (1 C), 126.06 (1 C), 125.88 (1 C), 125.50 (1 C), 124.92 (1 C), 124.46 (1 C), 123.51 (1 C), 122.75 (1 C), 122.64 (1 C), 122.19 (1 C), 119.23 (1 C), 117.69 (1 C), 115.87 (1 C), 115.12 (1 C), 114.53 (1 C), 45.03 (3 C), 31.11 (3 C). Elemental analysis, calcd for C₃₄H₂₅F₃N₄O₃S₃: C, 59.12%; H, 3.65%; N, 8.11%. Found: C, 60.06%; H, 3.28%; N, 8.46%.

### Example 5

Compound (12) was prepared by condensation of 1-methyl-2-pyrrolecarboxaldehyde with N-methylquinolinium triflate (4) with catalytic amount of piperidine. Compound (14) was obtained starting from compound (12) through a Vilsmeier type reaction, followed by condensation with (4), with catalytic amount of piperidine, according to the following scheme:

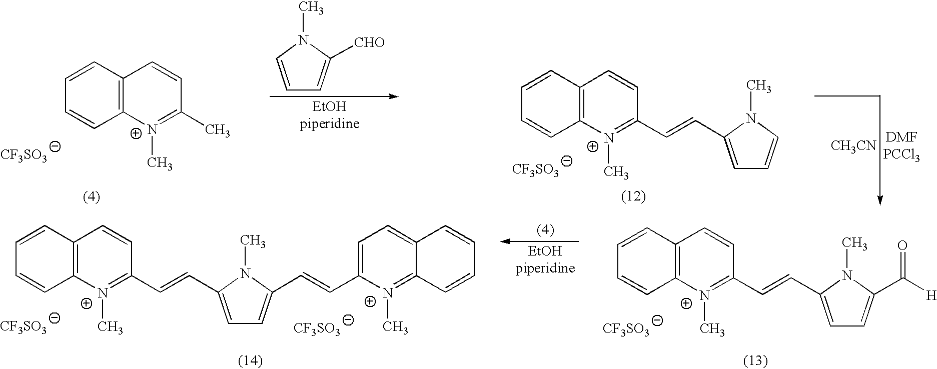

1-[N-methylquinol -2-yl]-2-(N-methyl-2-pyrrolyl)ethylene triflate (12)

A solution of (4) (2.000 g, 6.6 mmol) and 0.1 ml of piperidine in ethanol (10 ml) was added at room temperature to a solution of N-methyl-2-pyrrolecarboxaldehyde (0.731 g, 6.7 mmol) in 10 ml of the same solvent. The reaction mixture was stirred under reflux for 2 h and then cooled with an ice bath. A red precipitate was filtered under reduced pressure and washed with 3 ml of ethanol. The product was obtained as a red solid (1.826 g, 4.62 mmol, 70%): mp 208-210° C. ¹H-NMR (DMSO-d₆) 8.81 (1 H, d, J=9.1), 8.62 (1 H, d, J=9.1), 8.41 (1 H, d, J=9.1), 8.24 (1 H, d, J=9.5), 8.18 (1 H, d, J=15.25), 8.08 (1 H, t, J=7.1), 7.85 (1 H, t, J=7.5), 7.47 (1 H, d, J=15.25), 7.40 (1 H, d, J=3.95), 7.30 (1 H, m), 6.34 (1 H, m), 4.40 (3 H, s), 3.90.(3 H, s).

1-[N-methylquinol-2-yl]-2-(N-methyl-5-formylpyrrol-2-yl)ethylene triflate (13)

Freshly distilled POCl₃ (0.711 g, 4.64 mmol) was added dropwise, at −15° C. under nitrogen atmosphere, to anhydrous dimethylformamide (0.339 g, 4.64 mmol), the reaction mixture was diluted with anhydrous acetonitrile (4ml). A solution of 1-(N-methylquinol-2-yl)-2-(N-methylpyrrol-2-yl)ethylene triflate (12) (0.918 g, 2.32 mmol) in anhydrous acetonitrile (15 ml) was added dropwise at −15° C., then the reaction mixture was stirred at ambient temperature for 6 h, observing the formation of a precipitate that was filtered off under reduced pressure. The product was obtained as a red, fluorescent, precipitate (0.620 g, 1.46 mmol, 63%): mp 218-220° C. d. ¹H-NMR (DMSO-d₆) 9.71 (1 H, s), 9.08 (1 H, d, J=8.92), 8.76 (1 H, d, J=9.01), 8.57 (1 H, d, J=9.01), 8.36 (1 H, d, J=8.90), 8.21 (1 H, d, J=15.55), 8.19(1 H, t, J=7.64), 7.96(1 H, t, J=7.54), 7.93(1 H, d, J=15.53), 7.45 (1 H, d, J=4.41), 7.21 (1 H, d, J=4.31), 4.57 (3 H, s), 4.15 (3 H, s).

N-methyl-2,5-[1-(N-methylquinol-2-yl)ethen-2-yl]pyrrole triflate (14)

A solution of N-methyl-2-quinaldinium triflate (0.216g, 0.71 mmol) and a few drops of piperidine, in ethanol (15 ml), was added to a solution of (14) (0.300 g, 0.71 mmol) in ethanol (20 ml). The reaction mixture was kept under reflux for 30 min, and then cooled to allow separation of the product. The precipitate was filtered off under reduced pressure and washed with 4 ml of absolute ethanol. The product was obtained as a dark violet solid (0.258 g, 0.36 mmol, 52%). mp 307-308° C. ¹H-NMR (DMSO-d₆) 9.47 (2 H, d, J=9.1), 8.74 (2 H, d, J=9.1), 8.51 ( 2 H, d, J=9.3), 8.31 (2 H, d, J=9.3), 8.28 (2 H, d, J=15.35), 8.15 (2 H, t, J=7.55), 7.91 (2 H, t, J=7.55), 7.79 (2 H, d, J=15.25), 7.68 (2 H, s), 4.52 (6 H, s), 4.12 (3 H, s); ¹³C-NMR (DMSO-d₆) 155.36 (2 C), 142.74 (2 C), 139.25 (2 C), 137.17 (2 C), 134.62 (2 C), 133.51 (2 C), 129.92 (2 C), 128.66 (2 C), 127.48 (2 C), 120.77 (2 C), 119.15 (2 C), 117.21 (2 C), 116 .31 (2 C), 39.69 (2 C), 31.40 (1 C). Elemental analysis, calcd for C₃₁H₂₇F₆N₃O₆S₂: C, 52.03%; H, 3.80%; N, 5.87%. Found: C, 52.27%; H, 3.97%; N, 6.24%.

### Example 5

Compound (15) was prepared by alkylation of 4-methylquinoline with 2-bromoethanol in acetonitrile. Compound (16) was synthesized by condensation of compound (15) with compound (8) in ethanol, with catalytic amount of piperidine, according to the following scheme:

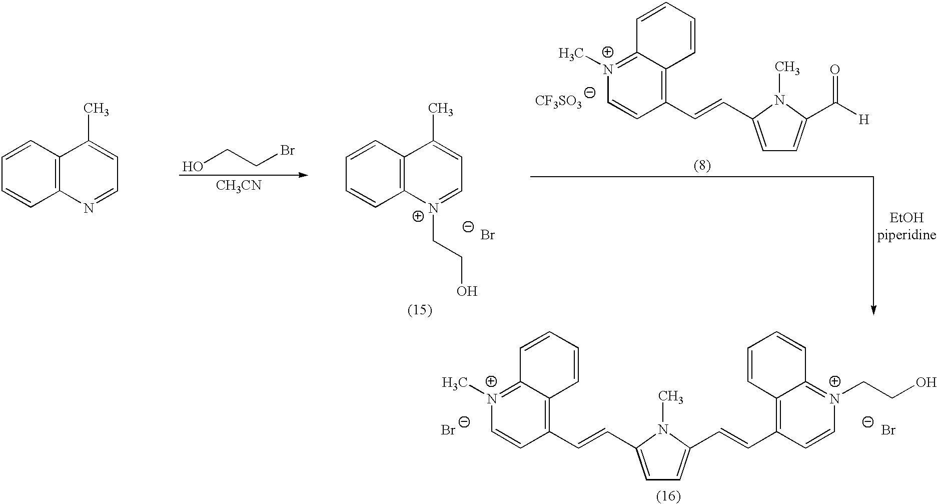

N-(2-hydroxyethyl)lepidinium bromide (15)

A solution of lepidine (0.280 g, 1.96 mmol) in acetonitrile (3 ml) was added to a solution of 2-bromoethanol (0.250 g, 2 mmol) in the same solvent (2 ml). The reaction mixture was kept under reflux for 2 h and then the solvent was evacuated under reduced pressure. The resulting white oil was treated with ethyl ether (3 ml) observing the separation of a precipitated that was filtered under reduced pressure and washed with toluene (2 ml). The product was obtained as a white solid (0.273 g, 1.02 mmol, 52%): mp 196-197° C.

N-methyl-2-[1-(N-methylquinol-4-yl)ethen-2-yl]-5-[1-[N-(2-hydroxyethyl)quinol-4-yl]ethen-2-yl]pyrrole bromide (16)

A solution of (15) (0.096 g, 0.36 mmol) in ethanol (8 ml) was added to a solution of (8) (0.152 mg, 0.36 mmol) in the same solvent (5 ml) and then a few drops of piperidine were added to the reaction as a catalyst. The mixture was stirred under reflux for 2 h and then cooled to allow precipitation of the product, that was filtered under reduced pressure and washed with ethanol (5 ml). The product was obtained as a blue solid (0.112 g, 0.18 mmol, 50%): mp ° C. ¹H-NMR (DMSO-d₆) 9.21 (1 H, d, J=6.75), 9.10 (1 H, d, J=6.70), 9.01 (2 H, d, J=8.60), 8.60 (2 H, d, J=6.69), 8.48 (1 H, d, J=8.97), 8.37 (1 H, d, J=8.85), 8.27 (2 H, d, J=15.3), 8.27 (1 H, t, J=7.10), 8.18 (2 H, d, J=15.28), 8.16 (1 H, t, J=7.08), 8.02 (1 H, t, J=7.52), 7.97 (1 H, t, J=7.26), 7.67 (2 H, s), 4.99 (2 H, t, J=5.5), 4.50 (3 H, s), 4.10 (3 H, s), 3.88 (2 H, t, J=5.5). Elemental analysis, calcd for C₃₀H₂₉Br₂ON₃*H₂O : C, 57.62%; H, 5.00%; N, 6.72%. Found: C, 57.67%; H, 5.79%; N, 6.40%.

### Example 6

The preparation of a glass film loaded with compound (3) in an host-guest type configuration is described in the following.

3-Glycidoxypropyltrimethoxysilane (3.780 g, 15.99 mmol) was added to a solution of (3) (0.005 g, 0.008 mmol) in methanol (1.6 ml), the reaction mixture was diluted with water (1 ml) and (3-aminopropyl)-triethoxysilane (0.218 g, 0.98 mmol) was added. The solution was stirred at room temperature for 65 min, observing a gradual increment in the viscosity. A few drops of the solution were deposed between 2 microscope slides and dried at room temperature for 2 days. FIG. 1 of the enclosed drawings shows the UV-visible absorption spectrum of a dried sol-gel film loaded with compound (3) in a host-guest type configuration, obtained according to Example 6.

Again, as an example, experimental data concerning the measurement of the optical limiting effect of the compound (3), prepared as a solution as described in the present invention, are provided as follows.

The following definitions are provided:

β (two-photon absorption coefficient; dependent on the concentration of the molecule). The β value can be experimentally determined by measuring the transmitted intensity I₁ as a function of the incident laser intensity I₀ using the following equation:

$$T = \frac{ln ( 1 + {I}_{0} L β )}{{I}_{0} L β}$$

where

$$T = \frac{{I}_{t}}{{I}_{0}}$$

and L is the thickness of the sample in units of cm.

The units of I₀ and Iₜ are I₀, It=[GW/cm²]; those for β are β=[cm/GWI].

It is useful to introduce a new parameter

$${β}^{′} = \frac{β}{c} ,$$

where c is the molar concentration of the sample, in units of [mol/l].

The molecular two-photon absorption cross-section σ₂ can be obtained from the measured value of β from the following equation:

$${σ}_{2} = \frac{β}{{N}_{0}} = \frac{β}{{N}_{a} c} {10}^{3} ,$$

where N₀ is the molecular density of the sample (in units of cm⁻³), Nₐ is the Avogadro's number and σ₂ is expressed in units of cm⁴/GW.

With reference to the plots of the figures in the enclosed drawings, FIGS. 2 and 3 show the transmittance and the output intensity, respectively, as a function of the input intensity. Values in FIGS. 2 and 3 were measured for compound (3) in the solvent DMSO (dimethylsulfoxide), using a 3×10⁻² M solution and a 790-nm laser radiation.

In FIG. 2, the value of transmittance T=1 corresponds to the situation of linear absorption, that is absence of optical limiting (the sample is fully transparent). The curve represents the best fit to experimental data using the relationship presented before, which correlates the transmittance T to the incident intensity through the two-photon absorption coefficient β. From the curve parameters it is possible to obtain the value of β, as reported below.

FIG. 2 clearly shows that it can be obtained a 90% optical limitation of the incident radiation (T=0.1), with a incident intensity of ca. 500 GW/cm².

FIG. 3 shows the optical power limiting response. The dashed line refers to the response of the solvent and thus corresponds, in other terms, to the linear transmission of the solution, that is the response that the sample would exhibit in absence of the nonlinear two-photon absorption process. In fact, at this wavelength the linear transmittance of the solution is T=1, that is the sample is fully transparent to low-intensity incident radiation.

With reference to the plots reported in FIGS. 2 and 3, a 3×10⁻² M solution of (3) in DMSO shows two-photon absorption coefficient β of 5.3×10⁻² cm/GW and a two-photon absorption cross-section σ₂ of 0.20×10⁻²⁰ cm⁴/GW. The optical limiting experiment was performed using a 790-nm laser radiation (repetition rate 10 Hz, pulse width pw=150 fs, sample thickness L=1 cm, divergence angle ca. 5 mm).

According to a further aspect of the present invention, in addition to the optical power limiting activity, the above described compounds are also indicated for other two-photon absorption based applications, such as two-photon laser scanning confocal fluorescence microscopy, where such systems behave as imaging agents.

1. A compound of the formula (I) 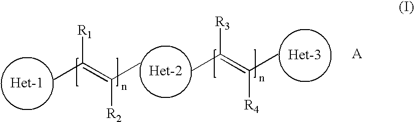
wherein het-1 and het-3 are identical or different, and are selected among the following heterocyclic groups: 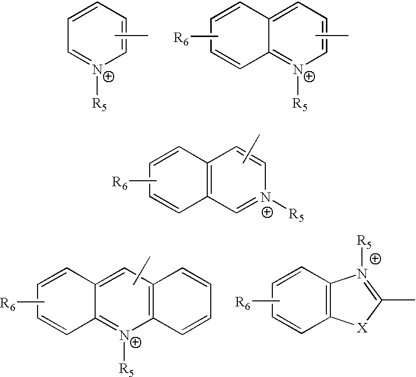
wherein X may be O, S or Se, and wherein R₅ and R₆ are the same or different, and are selected from the group consisting of H, alkyl groups having from 1 to 18 carbon atoms, alkoxy, aminoalkyl, alkylhalide, hydroxyalkyl, alkoalkyl, alkysulfide, alkylthiol, alkylazide, alkylcarboxyclic, alkylsulfonic, alkylisocyanate, alkylisothiocyanate, alkylalkene, alkylalkyne, aryl, and that can contain electronpoor ethenylic moieties such as maleimide, capable to react with nucleophilic groups such as —SH;
and Het-2 is selected among the following heterocyclic-groups: 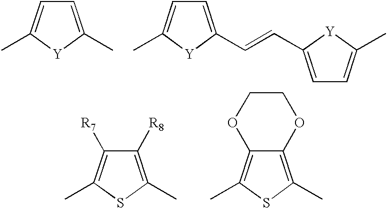
wherein Y may be O, S, NZ, wherein Z=H, lower alkyl, aryl; and R₇ and R₈ may be the same or different, and are lower alkyl, lower alkoxy or hydroxyalkyl;
wherein n=1, 2, and A is selected among the anions alkylsulfonate, arylsulfonate, triflate, halide, sulfate, phosphate;
and wherein R₁, R₂, R₃, R₄, the same or different, are indipendenty selected from the group of H, lower alkyl, alkoxyalkyl, aryl, cyano, alkoxycarbonyl, —(CR₉R₁₀)m-Het, wherein 0<m<10, R₉ and R₁₀, the same or different, are selected from the group of H, lower alkyl, and Het may be Het-1 or Het-2 or Het-3.

2. A compound of claim 1, having the following formula (3):

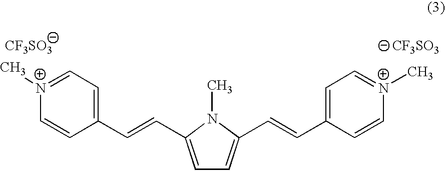

3. A compound of claim 1, having the following formula (5): 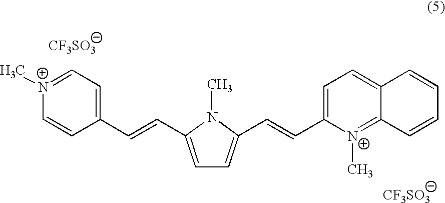
wherein the alkyl is selected among alkyl —(CH₂)₁—CH₃ with 1=1-18, and branched chain alkyl groups up to 19 carbon atoms; said aryl include aromatic hydrocarbon having 5 or 6 ring carbon atoms and heteroatomatic compounds having 5 or 6 ring atoms and containing 1 or 2 ring heteroatoms different than C, such as phenyl, toluyl, napthyl, furanyl, thiophenyl, pyrrolyl, pyridyl, and proviso the following compound is disclaimed from the said formula (I):

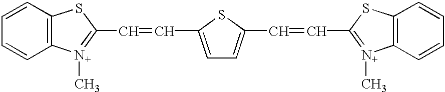

4. A compound of claim 1, having the following formula (9):

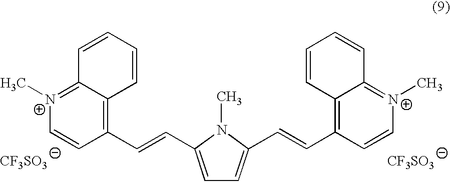

5. A compound of claim 1, having the following formula (11):

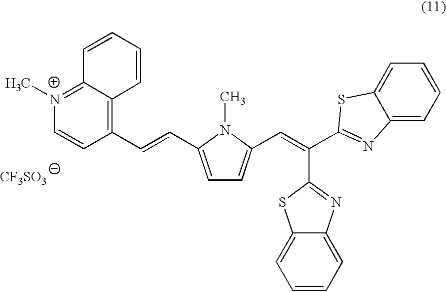

6. A compound of claim 1, having the following formula (14);

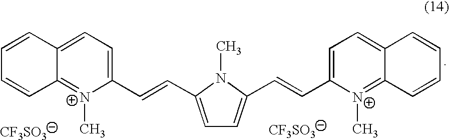

7. A compound of claim 1, having the following formula (16):

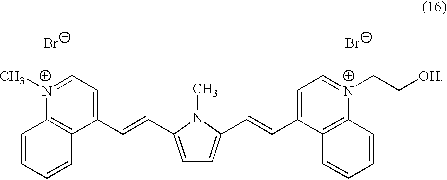

8. An intermediate compound for the synthesis of the compound of claim 2, having the following formula (2):

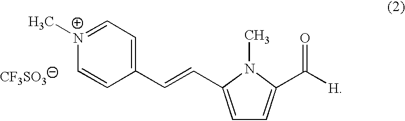

9. An intermediate compound for the synthesis of the compound of claim 3, having the following formula (4):

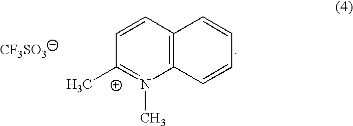

10. An intermediate compound for the synthesis of the compound of claim 4, having the following formula (6):

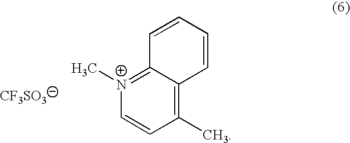

11. An intermediate compound for the synthesis of the compound of claim 4, having the following formula (7):

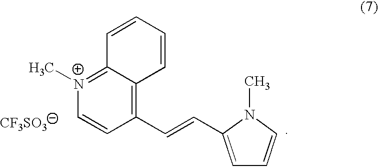

12. An intermediate compound for the synthesis of the compound of claim 4, having the following formula (8):

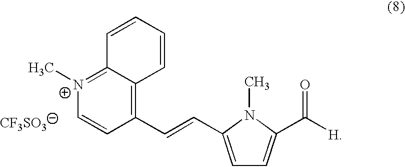

13. An intermediate compound for the synthesis of the compound of claim 6, having the following formula (12):

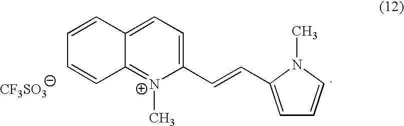

14. An intermediate compound for the synthesis of the compound of claim 6, having the following formula (13):

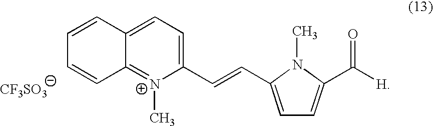

15. Two-photon absorbing cromophore characterized by being a compound of any of claims 1 to 7.

16. A compound according to any of claims 1 to 7 for use as optical power limiting agent via two-photon absorption.

17. A compound according to any of claims 1 to 7 for use as imaging agent with two-photon absorbing activity for application in two-photon laser scanning confocal fluorescence microscopy.

18. A composition comprising a compound according to claims 16 and 17 characterized by being prepared in solution or in a solid state.

19. A composition having optical power limiting activity characterized by the fact of comprising a compound according to claim 16.

20. A composition for use as imaging agent with two-photon absorption activity for application in two-photon laser scanning confocal fluorescence microscopy, characterized by the fact of comprising a compound according to claim 17, 16.

21. A composition according to claims 18, 19 and 20 characterized by the fact of comprising:
a polymer material chosen among poly(methacrylate), polyimide, polyamic acid, polystyrene, polycarbonate, polyurethane;
an organically modified silica (SiO₂) network.

22. A composition according to claim 21 characterized by the fact of being prepared as a film.

23. A composition according to claim 21 characterized by the fact of being prepared as a bulk.

24. Chromophore-functionalized polymer materials or organically-modified silica (SiO₂) network, prepared by condensation of a chromophore compound of general formula (1) and a polymer material which comprises poly(methacrylate), polyimide, polyamic acid, polystyrene, polycarbonate, polyurethane or an organically-modified silica (SiO₂) network.
